(19) United States
(12) Patent Application Publication
(10) Pub. No.: US 2024/0156888 A1
(43) Pub. Date: May 16, 2024

(54) SKIN EXTERNAL COMPOSITION AND METHOD FOR PREPARING THE SAME

(71) Applicant: AMOREPACIFIC CORPORATION, Seoul (KR)

(72) Inventors: Gayoung CHO, Yongin-si (KR); Yumi KANG, Yongin-si (KR); Jinsup SHIM, Yongin-si (KR); Lee Kyoung KWON, Yongin-si (KR); Mijin KIM, Yongin-si (KR); Yewon KIM, Yongin-si (KR); Byungfhy SUH, Yongin-si (KR); Sowoong CHOI, Yongin-si (KR)

(73) Assignee: AMOREPACIFIC CORPORATION, Seoul (KR)

(21) Appl. No.: 18/096,335

(22) Filed: Jan. 12, 2023

(30) Foreign Application Priority Data
Nov. 11, 2022 (KR) 10-2022-0150119

(51) Int. Cl. A61K 36/488; A61K 8/9789; A61K 8/98; A61K 9/06; A61K 35/644; A61K 36/734; A61K 36/8945; A61Q 19/00

(52) U.S. Cl. CPC A61K 36/488; A61K 8/9789; A61K 8/988; A61K 9/06; A61K 35/644; A61K 36/734; A61K 36/8945; A61Q 19/007; A61K 2236/11; A61K 2236/17; A61K 2236/331; A61K 2800/5922; A61K 2800/805

(57) ABSTRACT

One aspect of the present disclosure is to a skin external composition and a method for preparing the same, and more particularly, the skin external composition includes a Pueraria thunbergiana honey water extract as an active ingredient to exhibit excellent anti-inflammatory and moisturizing effects.

### TECHNICAL FIELD

This application claims the benefits of priorities based on Korean Patent Application No. 2022-0150119 filed on November 11, the entire contents of which are hereby incorporated by reference.

One aspect of the present disclosure relates to a skin external composition and a method for preparing the same.

### BACKGROUND ART

The skin, as a primary barrier of the human body, functions to protect internal organs from external environmental stimuli such as changes in temperature and humidity, ultraviolet rays, and pollutants, and undergo changes due to various internal and external factors with age. That is, the secretion of various hormones that regulate metabolism internally decreases, and the function of immune cells and the activity of cells decrease, resulting in a decrease in the biosynthesis of immune proteins and bio-structural proteins required for the body. In addition, externally, the content of ultraviolet rays reaching the ground of sun rays increases due to the depletion of the ozone layer, and free radicals, active harmful oxygen, and the like increase as environmental pollution further intensifies, thereby causing various changes such that skin thickness decreases, wrinkles increase, elasticity decreases, skin complexion becomes dull, skin troubles occur frequently, spots, freckles, and age spots also increase, complexion deteriorates, and skin tone darkens.

In order to protect or improve the skin against changes in the skin caused by such internal and external factors, various skin external compositions having anti-inflammatory and moisturizing effects have been developed.

In particular, interest in a skin external composition containing natural substances as active ingredients capable of minimizing side effects on the skin is growing.

In the prior art, it is disclosed to a preparing method of extracting main ingredients of natural substances by using honey as a solution or solvent and a composition prepared therefrom, and there is disclosed that the composition may exhibit excellent moisturizing and anti-inflammatory effects when used as a skin external preparation. At this time, animals and plants, fruits, oriental medicine, marine ingredients, mushrooms, and the like are disclosed as the natural substances.

Accordingly, there is a growing need for the development of a skin external composition using more various natural substances capable of exhibiting anti-inflammatory and moisturizing effects.

### DISCLOSURE

### Technical Problem

Accordingly, the present inventors have made many efforts to develop a skin external composition for anti-inflammatory and moisturizing effects including natural substances as active ingredients, and as a result, confirmed that a skin external composition including a Pueraria thunbergiana honey water extract as an active ingredient exhibited excellent anti-inflammatory and moisturizing effects.

Therefore, it is an object of one aspect of the present disclosure to provide a skin external composition including a Pueraria thunbergiana honey water extract as an active ingredient and a preparing method thereof.

### Technical Solution

To achieve the above object, one aspect of the present disclosure provides a skin external composition comprising a Pueraria thunbergiana honey water extract.

In addition, one aspect of the present disclosure provides a skin external composition may further including at least one selected from the group consisting of a Crataegus pinnatifida honey water extract and a Dioscorea opposita honey water extract.

In addition, one aspect of the present disclosure provides a skin external composition, wherein a weight ratio of the Crataegus pinnatifida honey water extract, the Dioscorea opposita honey water extract, and the Pueraria thunbergiana honey water extract is 1 to 7:1 to 7:1.

In addition, one aspect of the present disclosure provides a skin external composition, wherein the honey water extract is extracted with an extraction solvent containing honey and water.

In addition, the honey water extract includes an aged honey water extract or an unaged honey water extract, wherein the aged honey water extract is extracted after soaking and aging the extraction target in honey and mixing water, and the unaged honey water extract is extracted after soaking the extraction target in honey and mixing water without aging.

In addition, one aspect of the present disclosure provides a skin external composition, wherein a weight ratio of the honey and water is 1:1.2 to 20.

In addition, one aspect of the present disclosure provides a skin external composition, wherein the Pueraria thunbergiana honey water extract is included in an amount of 0.5 to 5 wt % based on the total weight of the composition.

In addition, one aspect of the present disclosure provides a skin external composition, wherein as a result of high-performance liquid chromatography (HPLC) analysis, the Pueraria thunbergiana honey water extract has a peak size of 0.05 A.U. or more between 15 to 25 mins., and the HPLC analysis result is measured by using a UV wavelength of 254 nm and H₂O and acetonitrile (ACN) as a mobile phase.

In addition, one aspect of the present disclosure provides a skin external composition used for anti-inflammatory.

In addition, one aspect of the present disclosure provides a skin external composition used for moisturizing.

Another aspect of the present disclosure provides a method for preparing a skin external composition including:
(S1) drying Pueraria thunbergiana; and
(S2) preparing a Pueraria thunbergiana honey water extract by soaking the dried Pueraria thunbergiana in honey, mixing water, and then extracting the mixture.

In addition, another aspect of the present disclosure provides a method for preparing a skin external composition, wherein in step (S2), the Pueraria thunbergiana honey water extract is prepared by soaking and aging the dried Pueraria thunbergiana in honey, mixing water, and then extracting the mixture.

In addition, another aspect of the present disclosure provides a method for preparing a skin external composition, further including mixing the Pueraria thunbergiana honey water extract with at least one selected from the group consisting of a Crataegus pinnatifida honey water extract and a Dioscorea opposita honey water extract.

### Advantageous Effects

A skin external composition according to another aspect of the present disclosure includes the Pueraria thunbergiana honey water extract as an active ingredient to exhibit excellent anti-inflammatory and moisturizing functions.

Further, the skin external composition includes the Crataegus pinnatifida honey water extract and the Dioscorea opposita honey water extract together with the Pueraria thunbergiana honey water extract to exhibit excellent anti-inflammatory and moisturizing functions.

### DESCRIPTION OF DRAWINGS

FIG. 1 is a graph showing the results of high-performance liquid chromatography (HPLC) analysis of extracts of Example 1 and Comparative Example 1.

FIG. 2 is a graph showing the results of HPLC analysis of extracts of Example 1, Example 2 and Comparative Example 1.

FIG. 3 is a graph showing results of HPLC ELSC (Evaporative Light-Scattering Detector) analysis of extracts of Example 1, Example 2 and Comparative Example 1.

### BEST MODE

Hereinafter, the present invention will be described in detail.

Skin External Composition

One aspect of the present disclosure relates to a skin external composition including a Pueraria thunbergiana honey water extract as an active ingredient.

The Pueraria thunbergiana BENTH means a flower bud of kudzu or a flower that has just begun to bloom, and is also called Pueraria lobata. The Pueraria thunbergiana blooms purple or purple in July to August, and has been reported to contain ingredients such as alkaloids, flavones, isoflavonoids, and saponin to have effects such as diabetes, liver protection, antioxidant, and cancer cell suppression, and contains a large amount of aromatic compounds to have a fragrant smell.

A Pueraria thunbergiana honey water extract prepared by extracting the Pueraria thunbergiana with honey and water may exhibit anti-inflammatory and moisturizing effects on the skin.

In an embodiment of one aspect of the present disclosure, the skin external composition may further include at least one selected from the group consisting of a Crataegus pinnatifida honey water extract and a Dioscorea opposita honey water extract.

The Crataegus pinnatifida has a fruit of Crataegus pinnatifida tree, a small tree belonging to the genus Malus, the family Rosaceae, called Crataegus pinnatifida or Crataegl Fructus, and is native to Asia and North America, and harder and much smaller than commonly planted apple trees, and has thorns. The Crataegus pinnatifida has a sour taste, but has a clear color and moderate sweetness to be made and eaten as jelly, canned food, alcohol, and tea, and in oriental medicine, an herbal medicine was used as a stomach medicine.

A Crataegus pinnatifida honey water extract prepared by extracting the Crataegus pinnatifida with honey and water exhibits anti-inflammatory and moisturizing effects on the skin, and particularly, may further enhance the anti-inflammatory and moisturizing effects on the skin by using the Pueraria thunbergiana honey water extract and/or the Dioscorea opposita honey water extract together.

In addition, the Dioscorea opposita is a tuberous root of Dioscorea japonica or Disocorea batatas belonging to the family Dioscoreaceae and has a cylindrical or unevenly cylindrical appearance and is sometimes cut vertically or horizontally. The outer surface thereof is milky white or yellowish white, and the bent surface thereof is flat, powdery or horny, and milky white. The fibre thereof is hard and easily broken. The Dioscorea opposita is collected and dried between the Sanggang (the first fall of frost) in autumn and the dongji, and the outer surface thereof is white or yellowish brown, and the inside thereof is hard with powdery or gelatinized keratin. The ingredients include starch, mucilage, protein, fat, arginine choline, and diastase. The Dioscorea opposita has almost no odor, has a sweet taste and is warm in nature.

A Dioscorea opposita honey water extract prepared by extracting the Dioscorea opposite with honey and water exhibits anti-inflammatory and moisturizing effects on the skin, and particularly, may further enhance the anti-inflammatory and moisturizing effects on the skin when used together with the Pueraria thunbergiana honey water extract and/or the Crataegus pinnatifida honey water extract.

In an embodiment of one aspect of the present disclosure, a weight ratio of the Crataegus pinnatifida honey water extract, the Dioscorea opposita honey water extract, and the Pueraria thunbergiana honey water extract may be 1 to 7:1 to 7:1.

Specifically, the weight ratio may be 1 to 7:1 to 7:1, 1 to 5:1 to 5:1, or 3 to 5:3 to 5:1. In the skin external composition, the Pueraria thunbergiana honey water extract alone may also be the active ingredient, but if the Crataegus pinnatifida honey water extract and/or the Dioscorea opposita honey water extract is included together, when the weight ratio is satisfied, the skin external composition may have the most anti-inflammatory and moisturizing effects without side effects.

In an embodiment of one aspect of the present disclosure, the honey water extract may be extracted with an extraction solvent containing honey and water. The extraction solvent may include honey and water in a mixed or diluted state.

In an embodiment of one aspect of the present disclosure, the honey water extract may include an aged honey water extract or an unaged honey water extract.

The aged honey water extract is extracted after soaking and aging an extraction target in honey and mixing water, and the unaged honey water extract may be extracted after soaking the extraction target in honey and mixing water without aging. The unaged honey water extract may also be referred to as a mixed honey water extract. The extraction target may refer to natural products used in one aspect of the present disclosure, that is, Pueraria thunbergiana, Crataegus pinnatifida, and/or Dioscorea opposita.

The aging in the extraction process does not greatly affect the composition of the finally prepared honey water extract.

In an embodiment of one aspect of the present disclosure, the weight ratio of honey and water used in the extraction process may be 1:1.5 to 20.

Specifically, the weight ratio may be 1:1.2 to 20, 1:1.2 to 15, 1:1.2 to 10, 1:1.2 to 5 or 1:1.5 to 4. If the weight of the water is smaller than the weight ratio range, extraction may not be performed efficiently, and if the weight of the water is greater than the weight ratio range, there may be no benefit due to excess weight.

In an embodiment of one aspect of the present disclosure, the Pueraria thunbergiana honey water extract may be included in an amount of 0.5 to 5 wt % based on the total weight of the composition.

Specifically, the content of the Pueraria thunbergiana honey water extract may be 0.5 wt % or more, 0.8 wt % or more, 1 wt % or more, or 1.2 wt % or more, and 2 wt % or less, 3 wt % or less, 4 wt % or less or 5 wt % or less. If the content of the Pueraria thunbergiana honey water extract is less than 0.5 wt %, the anti-inflammatory and/or moisturizing effect may decrease, and if the content exceeds 5 wt %, the feeling of use may decrease. At this time, even when the Crataegus pinnatifida honey water extract and/or the Dioscorea opposita honey water extract are/is included together with the Pueraria thunbergiana honey water extract, the total content thereof may be included within the above content range.

In an embodiment of one aspect of the present disclosure, the Pueraria thunbergiana honey water extract has a peak size of 0.05 A.U. or more between 15 and 25 minutes as a result of high-performance liquid chromatography (HPLC) analysis, and the HPLC analysis result may be measured by using a UV wavelength of 254 nm and H₂O and acetonitrile (ACN) as a mobile phase.

The HPLC analysis result of the Pueraria thunbergiana honey water extract may be commonly applied to the aged honey water extract of Pueraria thunbergiana and the unaged honey water extract of Pueraria thunbergiana.

Unlike a conventional Pueraria thunbergiana extract, it can be seen that the HPLC analysis result is specifically shown in the Pueraria thunbergiana honey water extract, which is related to the anti-inflammatory and moisturizing effects of the Pueraria thunbergiana honey water extract.

In an embodiment of one aspect of the present disclosure, the skin external composition may be used for anti-inflammatory to alleviate or improve skin itching.

In an embodiment of one aspect of the present disclosure, the skin external composition may be used for moisturizing to alleviate or improve dry skin.

When the skin external composition according to one aspect of the present disclosure is formulated in the form of cosmetics, the skin external composition may be formulated in the form of softening lotion, astringent lotion, nutrient lotion, eye cream, nutrient cream, massage cream, cleansing cream, cleansing foam, cleansing water, powder, essence or pack, and the formulation thereof is not particularly limited. In addition, the composition according to one aspect of the present disclosure may contain adjuvants commonly used in the field of cosmetology or dermatology, such as fatty substances, organic solvents, solubilizers, thickeners, gelling agents, softeners, antioxidants, suspending agents, stabilizers, foaming agents, fragrances, surfactants, water, ionic or nonionic emulsifiers, fillers, metal ions sequestering agents, chelating agents, preservatives, vitamins, blocking agents, humectants, essential oils, dyes, pigments, hydrophilic or lipophilic actives, lipid vesicles or any other ingredient commonly used in cosmetics. The adjuvant is introduced in an amount generally used in the field of cosmetology or dermatology. In addition, the composition of one aspect of the present disclosure may contain a skin absorption promoting substance in order to increase a skin improvement effect.

In the skin external composition as an aspect of one aspect of the present disclosure, the composition may be a skin external pharmaceutical composition.

The formulation of the pharmaceutical composition according to one aspect of the present disclosure may be a solution, a suspension, an emulsion, a gel, a drop, a suppository, a patch or a spray, but is not limited thereto. The formulation may be easily prepared according to a conventional method in the art, and may be prepared by appropriately using excipients, wetting agents, emulsification accelerators, suspending agents, salts or buffers for osmotic pressure control, coloring agents, spices, stabilizers, preservatives, preservatives, or other commonly used additives.

The active ingredient of the pharmaceutical composition of one aspect of the present disclosure will vary depending on the age, sex, weight, pathological condition and severity of a subject to be administered, a route of administration, or the judgment of a prescriber. The determination of the application amount based on these factors is within a level of those skilled in the art, and its daily administration dose may be, for example, 0.1 mg/kg/day to 100 mg/kg/day, more specifically 5 mg/kg/day to 50 mg/kg/day, but is not limited thereto.

Method for Preparing Skin External Composition

Further, one aspect of the present disclosure relates to a method for preparing a skin external composition.

The method for preparing the skin external composition according to one aspect of the present disclosure includes (S1) drying Pueraria thunbergiana; and (S2) preparing a Pueraria thunbergiana honey water extract by soaking the dried Pueraria thunbergiana in honey, mixing water, and then extracting the mixture.

The description of raw materials used in the method for preparing the skin external composition is as described above.

In one aspect of the present disclosure, in step (S1), Pueraria thunbergiana may be dried.

In one aspect of the present disclosure, in step (S2), the Pueraria thunbergiana honey water extract may be prepared by soaking the dried Pueraria thunbergiana in honey, mixing water, and then extracting the mixture.

In an embodiment of one aspect of the present disclosure, the Pueraria thunbergiana honey water extract may be prepared by soaking and aging the dried Pueraria thunbergiana in honey, mixing water, and then extracting the mixture.

When the Pueraria thunbergiana honey water extract is prepared, the Pueraria thunbergiana may be soaked in honey, not aged or aged, mixed with water, and then extracted. The Pueraria thunbergiana honey water extract prepared after aging may be referred to as an aged honey water extract of Pueraria thunbergiana, and the Pueraria thunbergiana honey water extract prepared after aging may be referred to as an unaged honey water extract of Pueraria thunbergiana.

In addition, the aging may be stored for 5 days to 15 days at a temperature of 20° C. to 30° C. Specifically, the aging temperature may be 20° C. or more, 22° C. or more, or 24° C. or more, and 26° C. or less, 28° C. or less, or 30° C. or less, and the aging time may be 5 days or more, 7 days or more, or 9 days or more, and 11 days or less, 13 days or less, or 15 days or less. If the aging temperature and/or aging time are less than the above range, the extract may not be sufficiently aged, and if the aging temperature and/or aging time exceed the above range, the efficacy of the extract may rather decrease due to over-aging.

In addition, the extraction may be ultrasonic extraction, chilling extraction, hot water extraction, reflux cooling extraction, or heat extraction.

In addition, the weight ratio of the Pueraria thunbergiana and the honey may be 1:2 to 30, 1:2 to 20, 1:2 to 10 or 1:2 to 5, and when the above range is satisfied, the extraction process proceeds smoothly, and excellent anti-inflammatory and/or moisturizing effects of the prepared Pueraria thunbergiana honey water extract may be exhibited.

In an embodiment of one aspect of the present disclosure, the method may further include mixing at least one selected from the group consisting of a Crataegus pinnatifida honey water extract and a Dioscorea opposita honey water extract with the Pueraria thunbergiana honey water extract.

The Pueraria thunbergiana honey water extract may be used alone, but may also be used together with the Crataegus pinnatifida honey water extract and/or the Dioscorea opposita honey water extract, and in this case, the respective honey water extracts may be prepared and then mixed. The Crataegus pinnatifida honey water extract and the Dioscorea opposita honey water extract may be prepared in the same manner as the Pueraria thunbergiana honey water extract.

Hereinafter, preferred Examples of one aspect of the present disclosure will be provided to help understand one aspect of the present disclosure. However, the following Examples are just illustrative of one aspect of the present disclosure, and it will be apparent to those skilled in the art that various changes and modifications can be made within the scope and the technical idea of one aspect of the present disclosure and that these variations and modifications are within the scope of the appended claims.

The Examples and Comparative Example below were performed according to compositions shown in Table 1 below.

TABLE 1

| | Weight ratio of raw material | | | Weight ratio of extract solvents | | |
|---|---|---|---|---|---|---|
| | extracts | | | (honey:water) | | |
| | Pueraria | Crataegus | Dioscorea | Pueraria | Crataegus | Dioscorea |
| | thunbergiana | pinnatifida | opposita | thunbergiana | pinnatifida | opposita |
| | honey | honey | honey | honey | honey | honey |
| | water | water | water | water | water | water |
| | extract | extract | extract | extract | extract | extract |
| Example 1 | 1 | — | — | 1:1.5 | — | — |
| Example 2 | 1 | — | — | 1:1.5 | — | — |
| Example 3 | 1 | 4 | — | 1:1.5 | 1:2 | — |
| Example 4 | 1 | — | 4 | 1:1.5 | — | 1:2 |
| Example 5 | 1 | 4 | 4 | 1:1.5 | 1:2 | 1:2 |
| Comparative | 1 | — | — | — (extract solvent:water) | | |
| Example 1 | | | | | | |

### Example 1: Preparation of Pueraria thunbergiana Honey Water Extract (Aged)

1 g of Pueraria thunbergiana was dried.

The dried Pueraria thunbergiana was soaked in 20 g of honey and aged at room temperature for 10 days, added with 30 g of water, and extracted through sonication to prepare a Pueraria thunbergiana honey water extract (honey:water=1:1.5).

### Example 2: Pueraria thunbergiana Honey Water Extract (Unaged)

A Pueraria thunbergiana honey water extract was prepared in the same manner as in Example 1, except that aging was not performed during the preparation of the Pueraria thunbergiana honey water extract.

### Example 3: Preparation of Pueraria thunbergiana/Crataegus Pinnatifida Honey Water Extract (Aged)

3-1. Preparation of Pueraria thunbergiana Honey Water Extract and Crataegus pinnatifida Honey Water Extract

A Pueraria thunbergiana honey water extract was prepared in the same manner as Example 1.

A Crataegus pinnatifida honey water extract was prepared by soaking 1 g of dried Crataegus pinnatifida in 2 g of honey, aging at room temperature for 10 days, adding 4 g of water, and then performing ultrasonic extraction (honey:water=1:2).

3-2. Mixing

The Pueraria thunbergiana honey water extract and the Crataegus pinnatifida honey water extract prepared in 3-1 above were mixed at a weight ratio of 1:4.

### Example 4: Preparation of Pueraria thunbergiana/Dioscorea Opposita Honey Water Extract (Aged)

4-1. Preparation of Pueraria thunbergiana Honey Water Extract and Dioscorea opposita Honey Water Extract

A Pueraria thunbergiana honey water extract was prepared in the same manner as Example 1.

A Dioscorea opposita honey water extract was prepared by soaking 1 g of dried Dioscorea opposite in 2 g of honey, aging at room temperature for 10 days, adding 4 g of water, and then performing ultrasonic extraction (honey:water=1:2).

4-2. Mixing

The Pueraria thunbergiana honey water extract and the Dioscorea opposita honey water extract prepared in 4-1 above were mixed at a weight ratio of 1:4.

### Example 5: Preparation of Pueraria thunbergiana/Crataegus Pinnatifida/Dioscorea Opposita Honey Water Extract (Aged)

5-1. Preparation of Pueraria thunbergiana Honey Water Extract, Crataegus pinnatifida Honey Water Extract and Dioscorea opposita Honey Water Extract

A Pueraria thunbergiana honey water extract, a Crataegus pinnatifida honey water extract and a Dioscorea opposita honey water extract were prepared in the same manner as Examples 3-1 and 4-1.

5-2. Mixing

The Pueraria thunbergiana honey water extract, the Crataegus pinnatifida honey water extract and the Dioscorea opposita honey water extract prepared in 5-1 above were mixed at a weight ratio of 1:4:4.

### Comparative Example 1: Preparation of Pueraria Thunbergiana Water Extract

1 g of Pueraria thunbergiana was dried.

The dried Pueraria thunbergiana was leached in 40 g of water and then extracted through sonication to prepare a Pueraria thunbergiana water extract.

### Experiment Example 1: HPLC Analysis

High-performance liquid chromatography (HPLC) analysis was performed on the Pueraria thunbergiana honey water extract (aged) of Example 1 and the Pueraria thunbergiana water extract of Comparative Example 1.

An HPLC analysis target was a sample in which the extracts of Example 1 and Comparative Example 1 were filtered and then not concentrated.

HPLC analysis conditions were performed at UV=254 nm for 50 min by using H₂O:ACN (acetonitrile) as a mobile phase.

In addition, as a control group, even with respect to honey (Control 1) obtained by mixing honey and water in the same ratio and separating a water layer, HPLC analysis was performed under the same conditions.

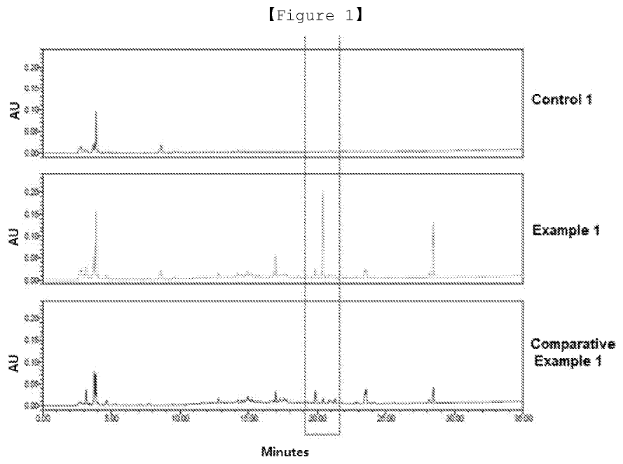

FIG. 1 is a graph showing results of HPLC analysis of extracts of Example 1 and Comparative Example 1.

Referring to FIG. 1, it can be seen that since both Example 1 and Comparative Example 1 show peaks at 20 min, the same ingredients are detected. However, in Example 1, the peak at 20 min was 0.20 A.U. or more, which was significantly larger than that of Comparative Example 1, and this was caused by the fact that honey and water were used together as the extraction solvent in Example 1.

Experiment Example 2: HPLC and HPLC ELSD Analysis

With respect to the Pueraria thunbergiana honey water extract (aged) of Example 1, the Pueraria thunbergiana honey water extract (unaged) of Example 2, and the Pueraria thunbergiana water extract of Comparative Example 1, high-performance liquid chromatography (HPLC) and HPLC ELSC (Evaporative Light-Scattering Detector) analysis were performed.

HPLC and HPLC ELSC analysis targets were samples obtained by filtering the extracts of Example 1, Example 2 and Comparative Example 1, concentrating the extracts, and then dissolving the extracts in methanol (MeOH).

HPLC and HPLC ELSC analysis conditions were performed at UV=254 nm for 50 min by using H₂O:ACN (acetonitrile) as a mobile phase.

In addition, as a control group, even with respect to honey E (Control 1) obtained by mixing honey and water in the same ratio and separating a water layer, adding the honey with EA, and then leaving the mixture at room temperature for 1 day, HPLC and HPLC ELSC analysis were performed under the same conditions.

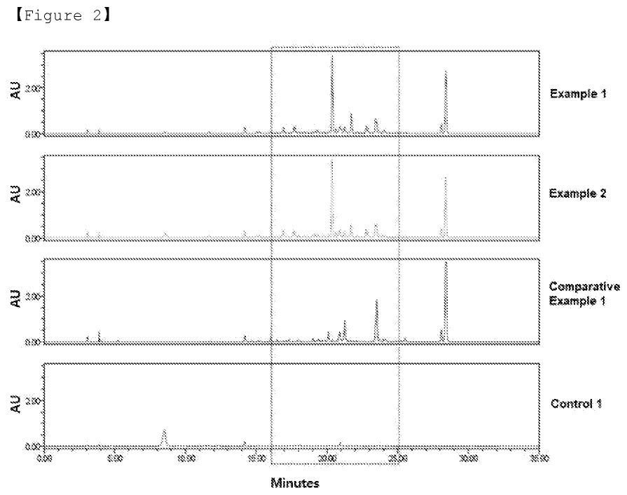

FIG. 2 is a graph showing results of HPLC analysis of extracts of Example 1, Example 2 and Comparative Example 1.

Referring to FIG. 2, it can be seen that since Example 1 and Example 2 show similar HPLC analysis results, aging in the extraction process does not significantly affect the composition of the prepared extract. In addition, it can be seen that since the peak at about 20 min is shown only in Examples 1 and 2 or significantly largely shown, this is caused by a difference depending on whether honey is used as an extraction solvent.

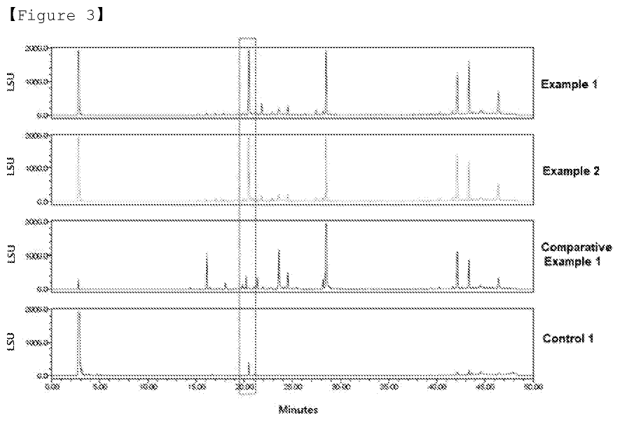

FIG. 3 is a graph showing results of HPLC ELSC (Evaporative Light-Scattering Detector) analysis of extracts of Example 1, Example 2 and Comparative Example 1.

Referring to FIG. 3, it can be seen that the peaks shown at about 20.40 min in Examples 1 and 2 are hardly confirmed or significantly small in Comparative Example 1 and Control 1. From this, it can be seen that the content of the ingredient corresponding to the peak significantly increases when honey water is used as an extraction solvent.

### Formulation Examples

Hereinafter, Formulation Examples of the skin external composition will be described, but only specifically described, not intended to limit the present invention.

TABLE 2
[Formulation Example 1] Lotion

| Mixed ingredients | Wt % |
|---|---|
| Pueraria thunbergiana honey water extract | 1.5 |
| 1-ascorbic acid-2-phosphate magnesium salt | 1.0 |
| Water-soluble collagen (1% aqueous solution) | 1.0 |
| Sodium citrate | 0.1 |
| Citric acid | 0.05 |
| Chelidonium majus extract | 0.2 |
| 1,3-butylene glycol | 3.0 |
| Purified water | To 100 |

TABLE 3
[Formulation Example 2] Cream

| Mixed ingredients | Wt % |
|---|---|
| Pueraria thunbergiana honey water extract | 1.0 |
| Polyethylene glycol monostearate | 2.0 |
| Self-emulsified monostearate glycerin | 5.0 |
| Cetyl alcohol | 4.0 |
| Squalene | 6.0 |
| Tri-2-ethylhexaneglyceryl | 6.0 |
| Sphingoglycolipid | 1.0 |
| 1,3-butylene glycol | 7.0 |
| Purified water | To 100 |

TABLE 4
[Formulation Example 3] Pack

| Mixed ingredients | Wt % |
|---|---|
| Pueraria thunbergiana honey water extract | 5.0 |
| Polyvinyl alcohol | 13.0 |
| 1-ascorbic acid-2-phosphate magnesium salt | 1.0 |
| Lauroylhydroxyproline | 1.0 |
| Water-soluble collagen (1% aqueous solution) | 2.0 |
| 1,3-butylene glycol | 3.0 |
| Ethanol | 5.0 |
| Purified water | To 100 |

TABLE 5
[Formulation Example 4] Cosmetic solution

| Mixed ingredients | Wt % |
|---|---|
| Pueraria thunbergiana honey water extract | 2.0 |
| Hydroxyethylene cellulose (2% aqueous solution) | 12.0 |
| Xanthan gum (2% aqueous solution) | 2.0 |
| 1,3-butylene glycol | 6.0 |
| Thick glycerin | 4.0 |
| Sodium hyaluronate (1% aqueous solution) | 5.0 |
| Purified water | To 100 |

TABLE 6
[Formulation Example 5] Ointment

| Mixed ingredients | Wt % |
|---|---|
| Pueraria thunbergiana honey water extract | 1.5 |
| Glycerin | 8.0 |
| Butylene glycol | 4.0 |
| Liquid paraffin | 15.0 |
| Beta Glucan | 7.0 |
| Carbomer | 0.1 |
| Caprylic/Capric Triglyceride | 3.0 |
| Squalane | 1.0 |
| Cetearyl glucoside | 1.5 |
| Sorbitan stearate | 0.4 |
| Cetearyl alcohol | 1.0 |
| Beeswax | 4.0 |
| Preservative,pigment,fragrance | Suitable amount |
| Purified water | Residual |

Although the present invention has been described with limited embodiments and drawings, it is to be understood that the present invention is not limited thereto, and various modifications and changes may be made within the technical idea of the present invention and the equivalent scope of the claims to be described below.

1. A skin external composition comprising a Pueraria thunbergiana honey water extract.

2. The skin external composition of claim 1, further comprising at least one selected from the group consisting of a Crataegus pinnatifida honey water extract and a Dioscorea opposita honey water extract.

3. The skin external composition of claim 2, wherein a weight ratio of the Crataegus pinnatifida honey water extract, the Dioscorea opposita honey water extract, and the Pueraria thunbergiana honey water extract is 1 to 7:1 to 7:1.

4. The skin external composition of claim 1, wherein the honey water extract is extracted with an extraction solvent containing honey and water.

5. The skin external composition of claim 1, wherein the honey water extract comprises an aged honey water extract or an unaged honey water extract, wherein the aged honey water extract is extracted after soaking and aging the extraction target in honey and mixing water, and the unaged honey water extract is extracted after soaking the extraction target in honey and mixing water without aging.

6. The skin external composition of claim 4, wherein a weight ratio of the honey and water is 1:1.5 to 20.

7. The skin external composition of claim 1, wherein the Pueraria thunbergiana honey water extract is comprised in an amount of 0.5 to 5 wt % based on the total weight of the composition.

8. The skin external composition of claim 1, wherein as a result of high-performance liquid chromatography (HPLC) analysis of the Pueraria thunbergiana honey water extract, a peak size between 15 to 25 min is 0.05 A.U. or more, and the HPLC analysis result is measured by using a UV wavelength of 254 nm and H₂O and acetonitrile (ACN) as a mobile phase.

9. The skin external composition of claim 1, wherein the skin external composition is used for anti-inflammatory.

10. The skin external composition of claim 1, wherein the skin external composition is used for moisturizing.

11. A method for preparing a skin external composition of claim 1 comprising:
(S1) drying Pueraria thunbergiana; and
(S2) preparing a Pueraria thunbergiana honey water extract by soaking the dried Pueraria thunbergiana in honey, mixing water, and then extracting the mixture.

12. The method for preparing the skin external composition of claim 11, wherein in step (S2), the Pueraria thunbergiana honey water extract is prepared by soaking and aging the dried Pueraria thunbergiana in honey, mixing water, and then extracting the mixture.

13. The method for preparing the skin external composition of claim 11, further comprising mixing the Pueraria thunbergiana honey water extract with at least one selected from the group consisting of a Crataegus pinnatifida honey water extract and a Dioscorea opposita honey water extract.
